(19) United States
(12) Patent Application Publication
(10) Pub. No.: US 2022/0265270 A1
(43) Pub. Date: Aug. 25, 2022

(54) SYRINGE STYLE BONE STAPLER INSERTER

(71) Applicant: ORTHO SOLUTIONS HOLDINGS LIMITED, Littleton, CO (US)

(72) Inventors: Kevin STAMP, Chapeltown Sheffield (GB); Dustin DUCHARME, Littleton, CO (US)

(21) Appl. No.: 17/667,902

(22) Filed: Feb. 9, 2022

Related U.S. Application Data
(63) Continuation-in-part of application No. PCT/US2020/046752, filed on Aug. 18, 2020.
(60) Provisional application No. 62/888,754, filed on Aug. 19, 2019.
(60) Provisional application No. 63/149,888, filed on Feb. 16, 2021.

(51) Int. Cl. A61B 17/064; A61B 17/068

(52) U.S. Cl. CPC A61B 17/0642; A61B 17/0682; A61B 2017/0645

(57) ABSTRACT

The invention is an inserter for a staple having a bridge member and opposing open legs and the inserter has a carriage, including a carriage body having at least one rail and a staple mount that holds the staple and provides a pair of camming members at the terminal end of a pair of deflectable or openable legs. The inserter has a syringe style deployment mechanism in which a finger pull is engaged by two fingers to draw the carriage upward on the rail and to cause the staple legs to meet the camming members such that the legs come into a transverse position. The invention further relates to an inserter system which includes the inserter and a drill guide.

### FIELD OF THE INVENTION

The invention comprises an inserter assembly for use with a room temperature superelastic U-shaped Nitinol staple intended for bone fixation. The inserter assembly has a hand activated vertical slide mechanism that in a first location along the longitudinal axis spreads the staple legs to transverse to allow for implantation and in a second location along the longitudinal axis disengages the staple from the inserter. By “vertical” it means along the inserter long axis which is generally up-right during implantation or the staple. The invention further relates to a drill guide for use with the staple and inserter, which has a retractor aspect, and which accepts a cartridge for the staple leg drill guide so as to allow the drill guide to be used with various configuration and size staples, including, for example two leg and four leg staples.

In addition, the drill guide and the inserter have a cooperating relationship which allows the drill guide cartridge to be slid out and the inserter to be slide in its place where the drill guide can be used as a jig for the proper alignment of the staple inserter to place the staple in the pre-drilled holes.

In a further embodiment of the syringe style inserter of the invention, the action of the inserter remains similar, but the mechanism for achieving it differs. The inserter still includes a carriage and attachable staple mount with terminal activation ramps but now the mount ramps can be opened and further is joined to the hand carriage by means of a rotatable self-tightening gripper. The staple mount can be dis-engaged to allow it to be used to re-mount a previously deployed staple, for example, in the event that a surgeon wishes to re-implant a staple for better seating in the bone.

### BACKGROUND OF THE INVENTION

Over 1.8 million orthopedic trauma fixation procedures were performed in the US in 2016, and the market is expected to reach over $4 billion by 2025. The fastest growing part of the market is the staple fixation segment, which is also expected to remain the fastest growing through to 2025. The primary drivers for growth are reportedly a reduced operating time as compared to screws, and plates.

While the state of the art has advanced the use of bone staples, there remain issues in the use and design of the deployment instrument or inserter. In particular, the inserter needs to be capable of single-handed use and needs to be able to deform the staple to cause the superelastic deformation and to inhibit over deformation, while permitting easy and reliable deployment of the staple in bone.

The invention also relates to a distractor that has a roller that spreads the flange ends of the drill guide and makes room for the staple drill guide cartridge that is captured in an appropriate keyed recess in the drill guide body. Upon removal of the drill guide cartridge, the inserter can be vertically slid into that position and the drill guide handle can be used for the proper alignment of the inserter.

This inserter design is intended for single use of the staple cartridge and multiple use of the remaining portion of the inserter so that cost is a consideration, drawing in materials and manufacturing methods that meet economic requirements while presenting a design that is sufficiently strong to reliably accomplish the job. This means that the inserter provides that the staple can be deformed to the insertion position with transverse legs, inserted into pre-drilled pilot holes and tamped into position across a bone divide, all in a design that is quick, reliable, and easy to use, and advantageously single-handedly.

In addition, the staple can be delivered in a sterile-packed cartridge, and advantageously with the staple in a relaxed position. The cartridge is subsequently loaded on the inserter which is used intraoperatively in a single handed and familiar syringe style activation mechanism to activate the staple by spreading the legs to the parallel position. This has the advantage of eliminating the storage and potential early deployment of an energized staple as well as not having a ‘fiddly” loading procedure for a staple that comes pre-packaged with a higher elastic energy.

In a further embodiment of the inserter, a rotatable gripper secures the staple cartridge or staple mount to the re-usable carriage. The carriage has a body comprising a terminal handle forming part of a handheld platform extending along a long axis into parallel rails that support a double ring finger pull mechanism. The body of the finger pull mechanism slides on the rails as the index and third finger draw it backward longitudinally along the rails toward the terminal handle which is pressed by the thumb pad of the user.

A set of arms extend in two directions outwardly from the rails to form a stop for the finger pull mechanism. When the inner surface of the flexed fingers of the user encounters the set of arms through a set of slots in the finger rings at a first position, the mechanism stops the user from pulling the finger pull mechanism any further back. At this point, the user switches the fingers to the distal facing surfaces of the double finger pull rings, and the arms are allowed to pass through slots in the proximal portions of the finger pull mechanism until detents inside the finger rings on either side of the rails encounter the set of arms in a second position. The carriage acts in cooperation with a pair of ramps on the ends of the staple mount legs to activate the legs of the staple to an open position when the finger pull mechanism is brought to the first position. When the finger pull mechanism is drawn to the second position, the staple can be deployed into pre-drilled holes in a bone or bones. In this embodiment, the inserter design allows the surgeon to remove the staple mount and open it to re-engage the staple legs to re-mount a staple for re-use during a surgical procedure.

### SUMMARY OF THE INVENTION

The invention provides an inserter assembly for a superelastic compressive bone staple. The staple has a bridge member that extends a length along an axis and which joins two or more legs spaced apart along the axis and is fabricated in a closed (converging legs) shape and is mechanically deformed or “activated” by the inserter during use to induce the superelastic shape memory properties. This allows the staple to compress bone segments in use.

The inserter comprises an activation assembly having a member, ram or carriage that has a sliding vertical or longitudinal cooperation with the rails of a ram housing mechanism or carriage body. In a first embodiment, the ram housing mechanism further has a staple clip and a stop lock and a staple cartridge having a terminal set of lateral shoulders that form a camming mechanism to splay the staple legs and a set of transverse medial shoulders which are engaged by the bottom surface of the staple bridge to open the staple clip legs and dis-engage the staple. During use, the staple lip is held between the ram housing mechanism and the rails as an upward vertical draw of the staple clip in the housing assembly relative to the staple acts to spread the staple legs on the lateral shoulders into transverse positions to open the legs and induce the superelastic properties for implantation.

The mechanism uses a hand activated ram or syringe type assembly which presses the member or ram downward relative to the slide housing mechanism, while the ram housing mechanism has a double set of finger holes which are used to draw the ram housing up-ward on the ram. Further, the ram includes a proximal tamp area (which is the same area contacted by the palm of the user's hand during the staple activation step) and a set of laterally extending curved draw stops for the finger holes of the ram housing. The finger holes have a first upper interior surface which moves upward relative to the ram as the tamp of the ram is pressed and the finger holes are drawn upward. The finger holes have a slotted upper area to allow the curved stops of the ram to slide through and contact the user's fingers. This stops the ram housing at the point at which the legs are opened to 90°. The staple is placed with the legs in alignment of a set of pilot holes for the staple legs and the user then grasps the lower exterior surface of the finger holes to draw the ram housing upward on the ram which is being pressed downward at the tamp portion of the ram. In this position, the bottom portion of the staple legs are disengaged from the shoulder ramp of the ram housing mechanism (i.e., specifically from the staple clip) and the staple is disengaged from the inserter.

A further embodiment of the inserter acts in the same manner to activate and deploy the staple but uses a syringe style mechanism with a differing connection between the carriage and staple mount to accomplish this task. In this embodiment, the inserter still includes a hand driven carriage (i.e., the ram housing and activation mechanism) which travels in the longitudinal direction along the rails of a body (i.e., the “ram”) to pull a detachable staple mount (i.e., the staple clip) towards the proximal end of the inserter. The carriage includes a slidable two finger ring pull mechanism for engagement by flexed index and third fingers as a terminal handle is cradled by the user's palm, to draw the carriage along the parallel rails of the body. The rails and staple mount are engineered to provide multiple longitudinally extending bearing surfaces to provide for the transfer of forces and to stabilize the carriage relative to the body and to ensure sufficient force to activate the staple without twisting or torquing the inserter.

The staple of the present invention is configured to accommodate fixation procedures in the forefoot, midfoot, rearfoot and hand, and the inserter (which can be disposable) allows implantation of the staple in bone in a surgical procedure so as to apply a compressive force across a division of bone segments. A preferable configuration for the bone staple is a substantially U-shaped staple, i.e., a staple having a transverse bridge member and downwardly extending legs (one or more pairs), which can be biased into a parallel “activated position” for insertion into the bone, and then released into a compressive configuration. Optionally, the staple bridge can include a configuration that assists in the interaction with the rails of the carriage body.

The inserter assembly or a portion of it such as the staple clip or staple mount, is advantageously pre-assembled (i.e. prior to surgery) with a “U-shaped” or modified “table top” style staple in a non-activated state (i.e. in which the legs converge toward each other, each at an angle of from 60° to 88°, and preferably at an angle from 70° to 85° relative to an axis along the bridge member of the staple) and includes an easy to use mechanism for “activating” the staple by deforming the legs to a transverse position and initiating the super elastic properties of the staple material. Advantageously, the staple bridge member is configured with a complex shape for engagement with a mating configuration of the inner surfaces of the staple mount legs.

Prior to the deployment of the staple, an inserter sub-assembly, the staple clip or staple mount, holds the staple so that it is constrained on the bottom side at the edges of the legs which ride against the shoulders of the slide housing mechanism. Prior to deployment, a ram handle is feed into the sliding housing or carriage mechanism which is held in a pair of recesses formed on lateral rails on the ram handle. At this point, the staple is also held on the top side by a pair of bosses on the distal end of the ram which advantageously fits within a recess within the bridge member defining separate legs on a lateral side. The staple member is also secured laterally against the inside of the legs of a staple clip member on one edge of the bridge member and against a distal end of the disengagement ram. In particular, the staple securing mechanism of the inserter includes integrated functional components including an expander with shoulders or camming members operatively connected to the disengagement ram which is that is operably held within the ram housing. The assembly includes a staple holding clip member (or staple mount) which includes a stop that is configured to lock into a correspondingly shaped opening in the slide housing. The slide housing has a laterally opposed pair of finger holes which can be used to draw the slide housing mechanism proximally on the rails while the ram is simultaneously pressed downward to lock into the lateral lock recesses and pull the staple legs open until the finger holes encounter a pair of curved stop members on the ram side. When the finger holes are drawn upward by engaging the bottom outer surface of the finger holes, the ram housing or carriage is moved past the first position until the ram housing mechanism pushes the staple off the inserter as the slide housing disengages the lateral lock recesses and the ram can be pushed downward to activate the staple.

The ram rails include a pair of outwardly extending flange members that are encountered by the user which provides a stop when the staple cartridge is being drawn upward on the slide housing mechanism. The ram rails can be pressed medially inward to open the distal end of the staple clip mechanism which opens the arms and disengages the staple from the inserter. As the staple cartridge is drawn upward by the slide handle housing, the camming surfaces on the distal shoulders of the clip mechanism are drawn upward and the staple legs are caused to splay apart.

In the second embodiment, the staple mount has a set of opposing openable distal legs which are joined in a pivot point and biased closed by a spring (similar to the action on a clothes pin) with an opposing set of proximal legs that are biased open. The staple bridge is embraced by corresponding configurations at the inner surfaces of the distal staple legs. At this end, the legs of the staple mount cooperate closed and include two laterally facing terminal ramp areas, along which the open end or ends of a two legged or four legged staple slide to cause the staple legs to open into a transverse position to be implanted into drilled holes during surgery. At the opposite ends and on the other side of the intermediate pivot point, the staple mount proximal legs each have a gripper recess that is bound on one face by a set of steps. These gripper recesses open to face away from each other, i.e., one facing the top of the staple mount, and the other facing the bottom of the staple mount.

In this embodiment, the carriage includes a gripper having a cylindrical body with a central bore. The body wall includes a pair of slots extending in the direction of the central axis from the distal edge of the gripper body, and each of the slots including a nubs which are spaced approximately 180° circumferentially around the gripper body. On the proximal side of each of the slots, there is a recess which accommodates the stepped end of the staple mount leg. The nub has a tip providing an edge that seats into a step of the staple leg mount to secure the staple mount and since both sides of the gripper includes a mirror image of the longitudinal slot and intermediate nub arrangement, the gripper acts on the two sets of steps of the staple mount legs which face in opposing directions. The carriage includes a set of at least two cooperating pins or spiraling grooves with the other forming a part of the mating surface on the finger pull base which rides on the inserter body rails. The groove spirals diagonally upward and proximally away in a helix from the recesses which house the stepped portion of the staple legs when the staple mount is in position in the carriage. Two opposing springs bias the gripper distally forward back relative to the finger pull ring body to ensure the relationship between the nubs and the staple mount steps tightens by urging the gripper into the staple mount as the finger pull ring is proximally engaged closer to the terminal handle of the inserter.

The finger rings project laterally outward from a cylindrical body of the finger ring pull member which includes internal cylindrically shaped surfaces that mate in a sliding relationship with rounded external surfaces on the two parallel rail portions of the carriage body rails. These rails have central slots that cooperate with central bosses on the top and bottom of the staple mount. The staple mount further includes four offset bosses having top bearing surfaces which ride within inward facing longitudinal recesses within the rails. The rails also include inwardly extending flange members that are captured between the proximal legs of the staple mount to help secure the staple mount to the carriage body. These flange members include opposing transverse interruptions which allow the staple mount to be opened by pinching the proximal set of staple mount legs closed while it is in position on the carriage body. This opens the distal set of staple mount legs to allow a staple to be reengaged on the inserter.

In the first embodiment, the finger mount body includes a set of distally extending arms with an opening between that receives the proximal legs of the staple mount and having an inwardly set of flanges that act to capture further capture the staple mount.

In both embodiments of the inserter, the carriage body includes a carriage lock to stabilize the carriage for attaching the staple cartridge or mount. The carriage lock includes a set of release surfaces which are contacted by the insides of the user's finger as they engage the finger rings pulls to draw the carriage toward the proximal end of the inserter. In the further embodiment, the carriage lock is a leaf spring clip having a J-lock and which has rounded prominences that extend laterally outward through slots in the carriage body rails. As the user flexes their two fingers against the inside of the finger rings to draw the carriage, the fingers also close toward each other to unlatch the nose of the carriage lock from the finger ring pull body. Opposing springs in the carriage base bias the carriage in a distal direction, and now the closure of the user's fingers overcome this biasing pressure. The user's pressure on the prominences causes the carriage J-lock to close inward such that the nose which is caught in an opening on the finger ring base escapes and frees the carriage to move along the body rails. The finger rings include openings at the proximal side so that the user's fingers are eventually contacted by a first set of carriage stops on the distal surfaces of wing members on the carriage body. During this length of travel, the staple mount is also drawn proximally, and the ends of the carriage body rails apply a distally directed force to the staple bridge which is transferred to the ends of the staple legs contacting the ramps on the staple mount legs. This force causes the staple legs to open to a transverse position so that the staple is activated for implantation into a corresponding set of holes in a bone or bones for implantation.

Now the user switches their finger position to the distal set of finger grip surfaces on the distal surfaces of the finger rings. The user continues to draw the finger rings proximally and the body rail wings slide through the openings of the finger rings until a stop within the finger rings is encountered by the rail wings. The staple mount is drawn further proximally, and the staple bridge encounter the medial join where the two staple legs are biased together by the spring of the staple mount. This causes the legs to open, and the staple is deployed from the staple mount into the pre-drilled holes.

In the event that the user is dissatisfied with the staple placement, and the user wishes to reseat the staple, the staple mount can be disengaged from the carriage. The staple can be removed, such as by tweezers from the bone, the staple mount legs can be opened to reload the staple onto the staple mount and the deployment procedure can be repeated.

In accordance with the invention, a staple is supplied preassembled (on a disposable inserter or inserter subassembly) as part of a sterile packed procedure kit. The staple is not pre-loaded, which means that the staple is not subjected to the mechanical deformation which initiates the superelastic characteristics of the staple. The staple inserter provides quick and efficient use with minimal user interaction which is accomplished by constraining the staple on the inserter on an expander component of the inserter and which expands the staple legs to a 90° insertion position. The staple is further captured on the inserter and prevented from inadvertent disassembly from the inserter by a ramp that acts as a cover in the resting or non-energized position.

The inserter retains the staple in a non-preloaded/non-energized position but interface with the staple in the proximal corners and at the terminal edges of the staple legs. This captures the staple securely on the inserter, and permits the activation of the staple for deployment. The design also enables use of the inserter with both symmetrical and asymmetrical leg staples.

The invention also relates to a drill guide that expands a set of tips laterally to expose a surgical site, and the drill guide body has a complex opening that forms a clip to secure a drill cartridge in the opening so that various pilot holes can be drilled using a single drill guide body.

### BRIEF DESCRIPTION OF THE DRAWINGS

FIG. 1 shows a top side perspective view of the staple inserter with a staple in accordance with the present invention;

FIG. 2 shows a view of the staple inserter with a staple of FIG. 1;

FIG. 3 shows a side cross sectional view about the lateral to medial plane of the inserter in accordance with FIG. 1;

FIG. 4 shows a cross-sectional sagittal side view of the distal portion of the staple inserter of FIG. 1;

FIG. 5 shows the sagittal cross-sectional view of the staple inserter of FIG. 1;

FIG. 6 shows a lateral edge detail of the staple inserter of FIG. 1 with a staple in a first position;

FIG. 7 shows the staple inserter of FIG. 1 with an activated staple from a front edge view;

FIG. 8 shows the staple inserter of FIG. 7 with a staple in a side view;

FIG. 9 shows a detail of the staple inserter of FIG. 1 with a relaxed staple in a bottom side view;

FIG. 10 shows a bottom side perspective view of the staple inserter drill guide with a four-legged staple in accordance with a second embodiment of the present invention;

FIG. 11 shows a bottom detail of the staple inserter and staple of the present invention;

FIG. 12 shows a bottom side detail of the staple deployment are of the staple inserter of FIG. 10;

FIG. 13 shows a detail view of the staple inserter ram of FIG. 10 without a staple;

FIG. 14 shows the full assembly of the staple inserter ram and staple clip of FIG. 10 and with the ram in a first position relative to the ram housing:

FIG. 15 shows staple clip of FIG. 10;

FIG. 16 shows a bottom side view of the finger holder of the staple inserter of FIG. 10;

FIG. 17 shows a top side view of the finger holder of the staple inserter of FIG. 10;

FIG. 18 shows a front side view of the staple ram of FIG. 10;

FIG. 19 shows a side view of the staple ram of FIG. 17;

FIG. 20 shows a top front view of the staple inserter and drill guide handle assembly of FIG. 10;

FIG. 21 is a front side view of a drill guide/compression device for use with the staple inserter system of the present invention;

FIG. 22 is a view of the drill guide/compression device assembly with the staple inserter of the present invention;

FIG. 23 is detail of the drill guide/staple inserter assembly used together in accordance with the present invention;

FIG. 24 is a side end view of a staple mount taken from the distal end in accordance with a second embodiment of the inserter of the invention;

FIG. 25 is a side end view of the second embodiment of the staple inserter including staple mount of FIG. 24;

FIG. 26 is a side end view of a staple mount of FIG. 24 taken from the proximal end;

FIG. 27 is a side end view of the staple inserter including staple mount of FIG. 25 taken from the proximal end;

FIG. 28 is a side end view of the staple mount of FIG. 24 with a cut away to show the connection of the staple bridge with the staple mount distal legs;

FIG. 29 is a side end view of the staple inserter of FIG. 25 having a staple mount cut away to show the use with a two legged staple and with the gripper removed to show the details of the finger ring pull mechanism;

FIG. 30 is a side end view of the staple inserter of FIG. 25 with the carriage drawn proximally to the first stop to activate the staple legs; and

FIG. 31 is a side end view of the staple inserter of FIG. 25 with the carriage drawn proximally to the second stop deploy the staple during implantation.

### DETAILED DESCRIPTION OF THE INVENTION

The present invention comprises an inserter 50 for use with a room temperature superelastic Nitinol compression staple 10 for bone fixation in the surgical management of fractures and reconstruction of the foot and hand. Typically, the staples used with the present invention have a nominally U-shaped profile with a bridge member 14 spanning a space between opposing legs 12 (and it should be understood that the present inserter is also suitable for use with a staple having four legs in which each end of the bridge member includes a pair of legs, or alternatively, the staple could have three legs with a pair on one end, and a single leg opposing the pair). The inserter 50 of the invention is illustrated herein with a staple having two opposing pairs of legs 12 that are separated by an opening 13 that extends into the bridge member 14 of the staple. This feature is used for the stabilization of the staple in the inserter 50.

The staple 10 has two or more, and preferably 2, 3, or 4 transversely extending legs 12 that will engage bones or bone segments through the cortical surfaces. The legs 12 are spaced apart from each other and joined together by bridge member 14 that extends across the area between legs at either end of the bridge member 14. As shown, the legs are joined to transitional extensions 16 which fold or curve at an angle of from 75° to 90°, and preferably from 85° to 90°.

The bridge member 14 has a top surface 20 and a bottom surface 22 which have corresponding shapes so that they are separated by a constant thickness for at least a portion, and preferably for at least 50%, and more preferably for at least 75% or even 90% of the surface area has a complex curving configuration. It extends along an axis preferably in a straight profile, but with a topography that curves in two dimensions. The shape includes two side edges, which may have an inwardly curving shape or may be represented by straight lines.

The staple is comprised of a material is elastic and has the ability to recover an original un-deformed shape so as to apply a compressive force. An example of a suitable material is a superelastic material which is activated into the superelastic state by mechanical deformation.

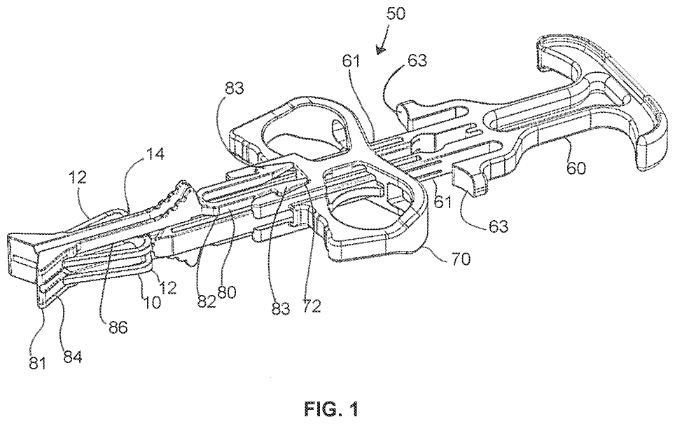

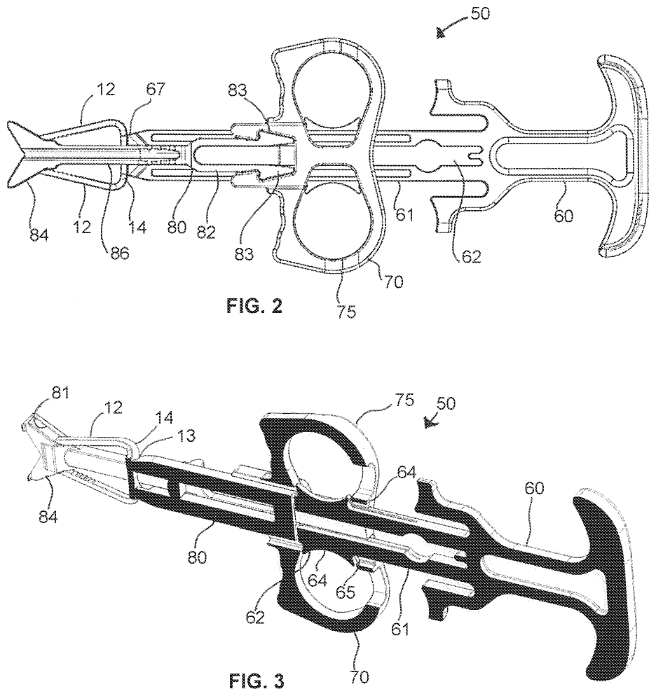

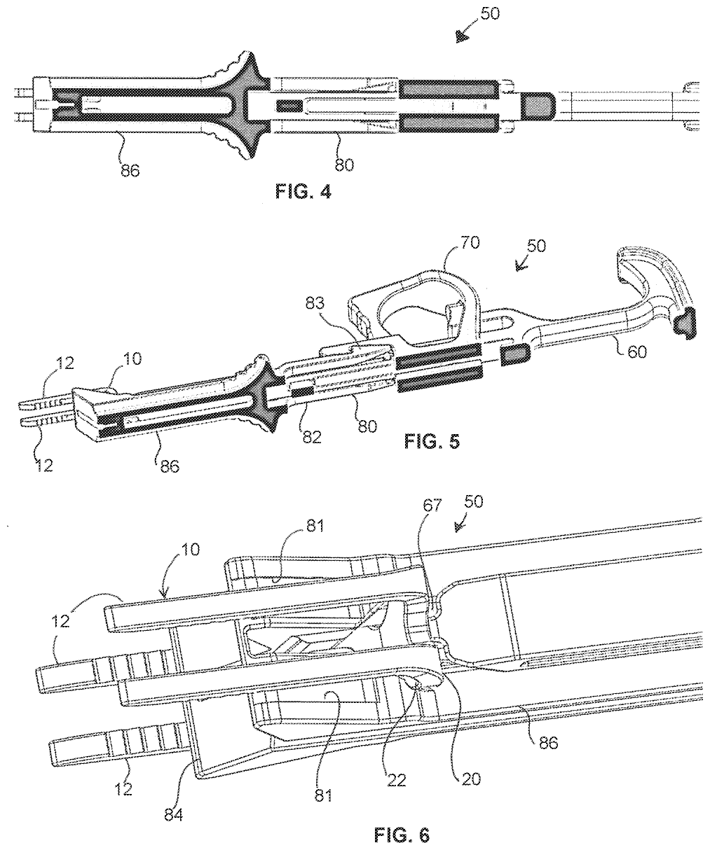

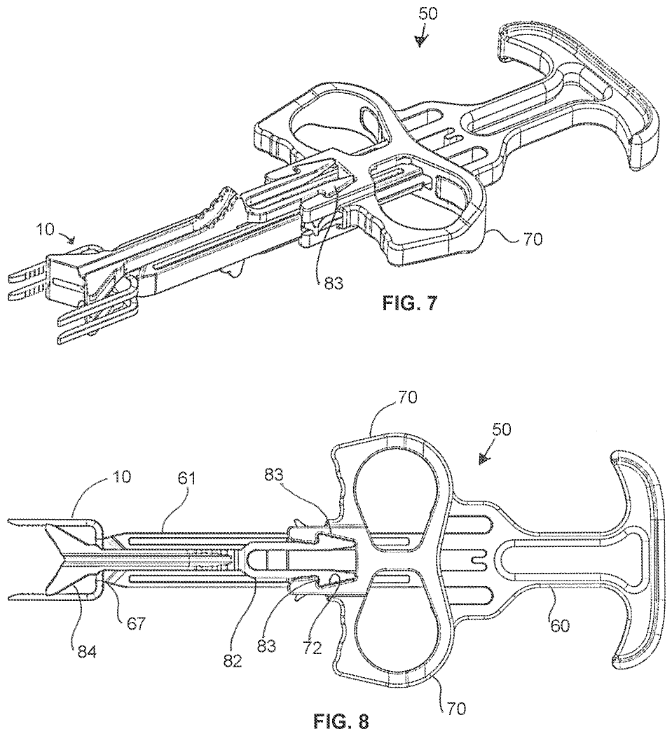

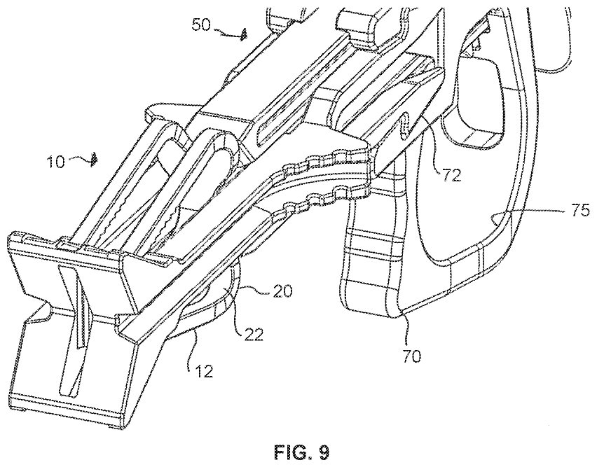

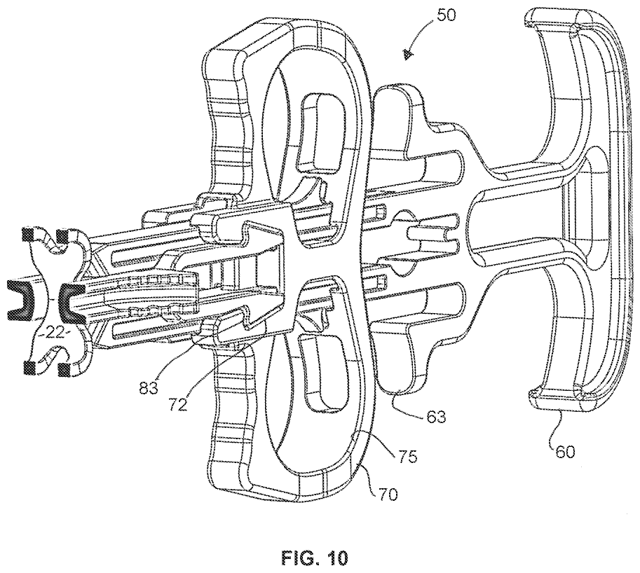

The inserter 50 comprises an assembly having a ram 60 that has a sliding vertical (i.e., along the long axis of the inserter) cooperation with ram housing mechanism 70. The ram housing mechanism has a staple clip 80 having a press point 82 and a terminal set of shoulders 84 that form a camming mechanism. The shoulders also have medial inner edges 81 that hold the staple on the staple clip. The press point 82 of the staple clip 80 is joined to a pair of pronged arms 83 which are held in a corresponding trapezoidal recess 72 in the ram housing mechanism as can be seen in FIG. 1.

The ram has a pair of longitudinal rail members 61, having a central slot 62. The rail members 61 also have a pair of laterally extending detent flanges 64 with openings 65 that hold stay members (not shown) on the finger hole portion 72 of the ram housing. The stay members can be drawn laterally out of the openings 65 using the finger holes 72 on the ram housing 70. This allows the ram 60 to be slide downwardly on the ram housing 70. This causes the staple clip 80 to move relative to the ram 60 which includes a pair of bosses 67 which engage the openings 13 in the bridge of the staple 10. The staple clip also includes a pair of legs 86 which terminate in the shoulders 84 that form a camming mechanism to spread the staple legs. When the staple is caught between the bosses 67 of the ram 60 and the shoulders 84 on the legs 86 of the staple clip 80, the staple legs 12 are extended to a transverse position and the staple is ready for insertion.

In a further embodiment, the inserter 200 includes a hand driven carriage 210 which slides in direction of the long axis along the rails of a body 220 (i.e. the “ram”) to pull a detachable staple mount 230. The carriage 210 includes a slidable handle 212 having a two finger ring pull 214 for an index finger and third finger as a terminal handle 216 is cradled by the user's palm to draw the carriage 210 along the parallel rails 222 of the body 220. The rails 222 and staple mount 230 are engineered to provide multiple bearing surfaces 233 to provide for the transfer of forces and to stabilize the carriage 210 relative to the body 220 and to ensure sufficient force to activate the staple 198, 198′ without twisting or torquing the inserter 200.

The staple mount 230, 232′ (corresponding differing elements of the four legged staple version having the same reference numeral with a ′) has a set of opposing openable distal legs 232, 232′ which are joined in a pivot point 234 and biased by a spring 236 into a closed position and in which the staple bridge is embraced by corresponding configurations at the inner surfaces 238, 238′ of the staple legs 232, 232′. At this end, the legs 232, 232′ of the staple mount 230, 230′ cooperate closed and include two ramp areas 235, 235′ along which the open end or end of a two legged 198 or four legged staple 198′ slide to cause the distal legs 232, 232′ to open into a transverse “activation” position to be implanted into drilled holes during surgery. At the opposite ends and on the other side of the intermediate pivot point 234, 234′, the proximal staple mount legs 237, 237′ each have a gripper recess 242 that is bound on one face by a set of steps 244. These recesses 242 open to face away from each other.

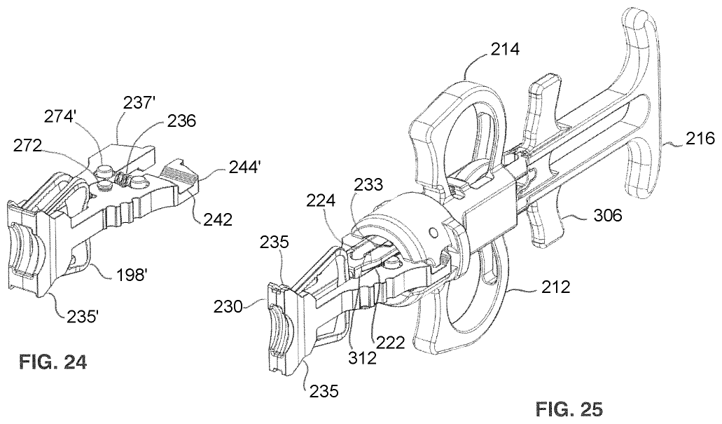

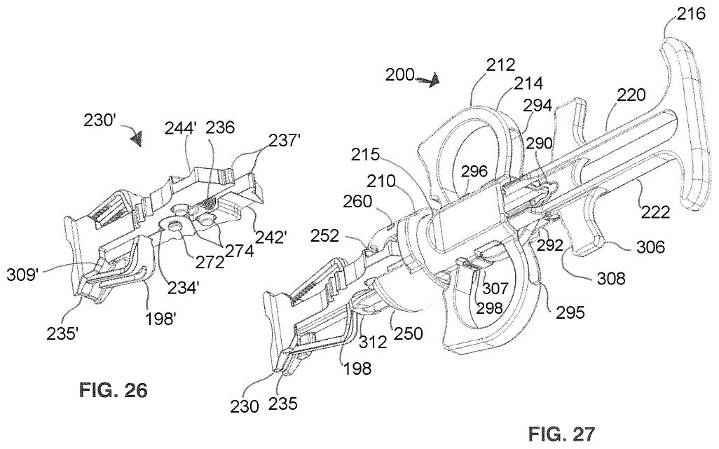

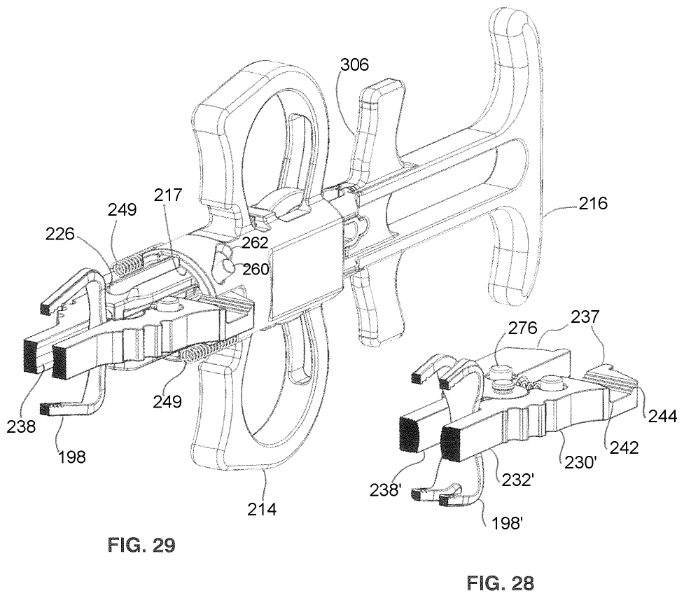

In the embodiment shown in FIGS. 24-27, the carriage 210 uses a rotatable gripper 250 to secure the staple mount 230. The gripper 250 has a pair of nubs 252 located in compound recesses 253 which are spaced approximately 180° circumferentially around the gripper body 251 which is advantageously cylindrical. The nubs 252 are formed in the wall of the gripper body 251 intermediate to a longitudinal slot 254 which ends in a recess 256 which accommodates the stepped end of the staple mount leg. The nub 252 has a tip 257 that seats into a step 244 of the staple leg mount to lock into the staple mount 230. Since opposing lateral surface of the gripper 250 have mirror image longitudinal slot 254 and intermediate nub 252 arrangements, the gripper 250 acts on the two sets of steps 244 of the staple mount legs 232 which face in opposing directions.

The carriage 210 includes a set of at least two cooperating pins 260 or spiraling grooves 262 with the other forming a part of the mating surface on the cylindrical finger pull base 215 of the moveable handle 212 which rides on rails 222 of the inserter body 220. The groove 262 spirals diagonally upward in a helix away from the stepped portion 244 of the mating proximal staple leg 237 when the staple mount 230 is in position in cooperation with the carriage 210. Two opposing springs 249 bias the gripper 250 proximally away relative to the finger pull rings but are compressed as the handle is drawn proximally manually using the finger pull rings. As the springs 249 are compressed the nubs 252 are rotated by the helical groove and pin mechanism into the staple mount steps 244 to tighten the carriage grip on the staple mount and compensate for the force exerted to open the staple legs.

The finger rings 214 have a body 215 which includes internal cylindrically shaped surfaces 217 that mate in a sliding relationship with rounded surfaces 224 on the two parallel rail portions of the carriage body rails 222. These rails 222 have central slots 226 that cooperate with central bosses 272 on the top and bottom of the staple mount 230. The staple mount 230 further includes four offset bosses 274 having top bearing surfaces 276 which ride within inward facing longitudinal recesses 278 within the rails 222. The rails 222 also include inwardly extending flange members 282 that are captured between the proximal legs 237 of the staple mount 230. These flange members 282 include opposing transverse interruptions (not shown) which allow the staple mount 230 to be opened while it is in position on the carriage body 220 to allow a staple 198, 198′ to be reengaged on the inserter 200.

As illustrated in FIGS. 24-27, the carriage 210 has a carriage lock 290. In this case, the carriage lock 290 is a leaf spring clip, which has rounded prominences 292 that extend laterally outward through slots 294 in the carriage body rails 222 as well as through lateral slots 295 in the body of the finger ring pull member 214. Thus, as the user flexes their two fingers against the inside of the finger rings 214 to draw the carriage 230, the fingers also close toward each other to unlock the J-lock. The carriage lock 290 biases and the carriage in a distal direction and has a J-lock to hold it in a locked position.

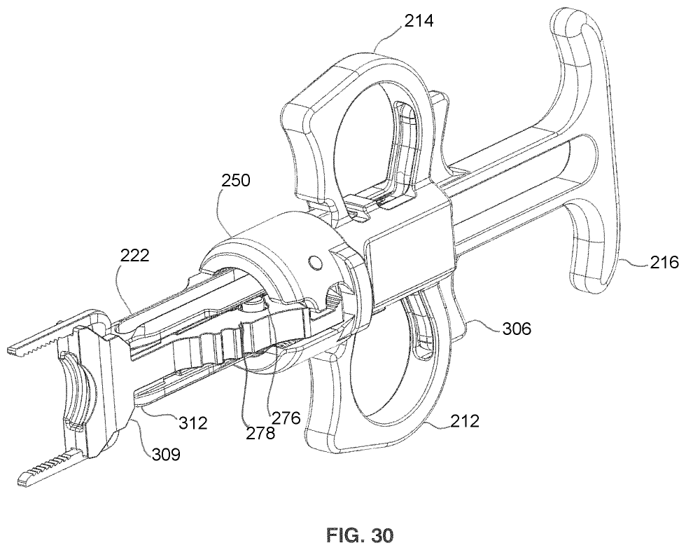

During use, the user's fingers overcome this biasing pressure. The user's pressure on the prominences 292 cause the carriage J-lock 296 to close inward such that the nose 298 which is caught in an opening 299 on the finger ring base escapes and frees the carriage 210 to move along the body rails 222. The finger rings 214 again include openings 295 at the proximal side so that the user's fingers are eventually contacted by a first set of laterally extending carriage stops 308 on the distal surfaces of wing members 306 on the carriage body. During this length of travel, the staple mount 230 is also drawn proximally, and the ends of the carriage body rails 222 apply a distally directed force to the staple bridge which is transferred to the ends of the staple legs 232 contacting the ramps 235 on the staple mount legs 232. As illustrated in FIG. 30, this force causes the staple legs to open to a transverse position so that the staple is activated for implantation into a corresponding set of holes in a bone or bones for implantation. In this position, the staple is introduced into a set of pre-drilled bone holes.

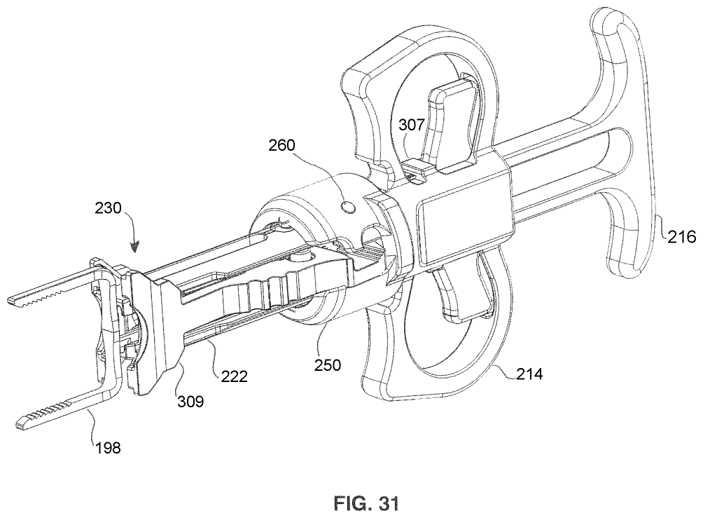

Now the user switches their finger position to the distal set of finger grip surfaces on the distal faces of the finger rings 214 of the moveable handle 212. The user continues to draw the carriage 210 proximally and the body rail wings 306 slide through the openings of the finger rings 214 until a detent 307 within the finger pull rings is encountered on each side of the long axis. The staple mount 230 is drawn further proximally, and the distal surface of the staple bridge encounters sloped areas 309 just proximal to the ramps of the staple mount and the medial join where the two staple legs are biased together by the spring 236 of the staple mount 230. In addition, a pair of shoulder members 312 sloping upward away from the distal abutment face of the rails meets and inner facing surface on the ramped area of the staple mount 235, 235′ to further bias the distal legs 232, 232′ of the staple mount open laterally. This causes the distal legs 232, 232′ to open as is shown in FIG. 31 and the staple is fully released from the staple mount as it is held in the pre-drilled holes.

In the event that the user is dissatisfied with the staple placement, and the user wishes to reseat the staple, the staple mount 230, 230′ can be disengaged from the carriage 210. The staple 198, 198′ can be removed, such as by tweezers from the bone, the staple mount legs 232, 232′ can be opened to reload the staple 198,198′ onto the staple mount 230,230′ and the deployment procedure can be repeated.

The staple of the present invention is configured to accommodate fixation procedures in the forefoot, midfoot, rearfoot and hand, and the inserter (which can be disposable) allows implantation of the staple in bone in a surgical procedure so as to apply a compressive force across a division of bone segments. A preferable configuration for the bone staple is a substantially U-shaped staple, i.e. a staple having a transverse bridge member and downwardly extending legs (one or more pairs), which can be biased into a parallel “activated position” for insertion into the bone, and then released into a compressive configuration.

The drill guide/compression instrument 150 of the present invention comprises an assembly of a handle member 160 having an elongated handle 161 which is shaped having straight or scalloped edges to accommodate being grasped while a compression/tension mechanism 170 includes a turn member 172 on a transverse screw 174. The handle member 160 extends into a first drill guide leg member 162 that remains stationary relative to the handle 161 and a second drill guide leg member 164 that opens and closes across a lateral gap by means of the distraction mechanism. More precisely, the user can open and close the gap between the legs by turning the turn member 172 which moves on the transverse screw 174 to drive the second leg closer to or farther away from the first leg.

Each of the first and the second leg include a transversely extending extension 175, 176 which has a cannulation 177 for a k-wire. Thus, the k-wires can be driven into the bone or bone segments, and the drill guide/distractor can be used to position or apply compression to the bone or bone segments, either before or after the drill guide is used to drill pilot holes for the legs of the associated bone staple.

Accordingly, one of the drill guide legs, here, the first leg, 162 includes a complex recess 178 that allows a drill guide cartridge 180 to be inserted into the drill guide/compression assembly. The drill guide cartridge includes a finger handle 182, and template member 182 that include spaced holes 184 according to various staple configurations. The drill guide cartridge also has a boss that cooperates with the complex recess to securely hold the cartridge in place relative to the handle member 160. The assembly can also include a compression cartridge 180 that can be inserted in place of the drill guide cartridge to help align and stabilize the staple at the user's discretion.

The operation of the inserter 50 for implantation of the compression staple is described as follows:

First the bone site is prepped by excising the site to access the relevant bones. Guide wires or more particularly, k-wires, can be placed into the bone segments, and the drill guide 50 is placed over the guide wires and the drill guide cartridge 80 is inserted for the proper placement of the pilot holes for the staple legs after the drill guide cartridge is removed, the drill guide handle and compressor accepts the inserter rails substituted in the position of the drill guide cartridge.

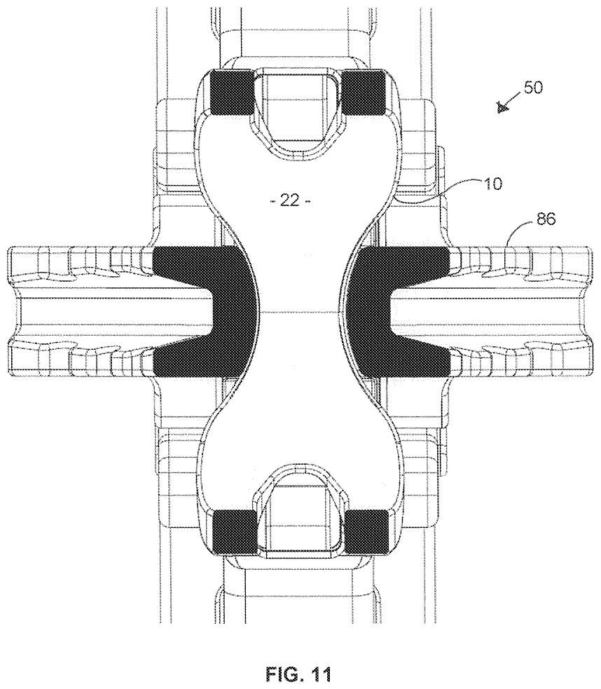

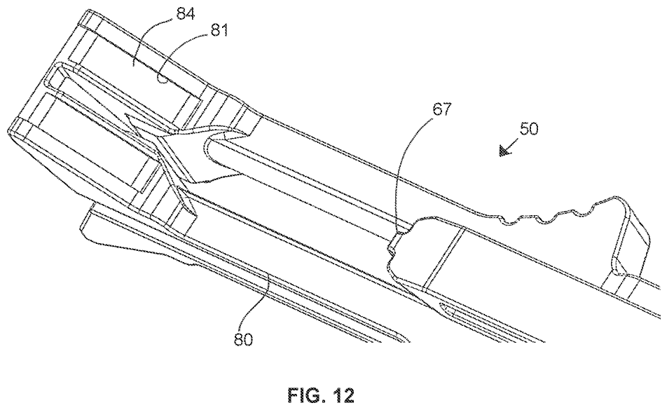

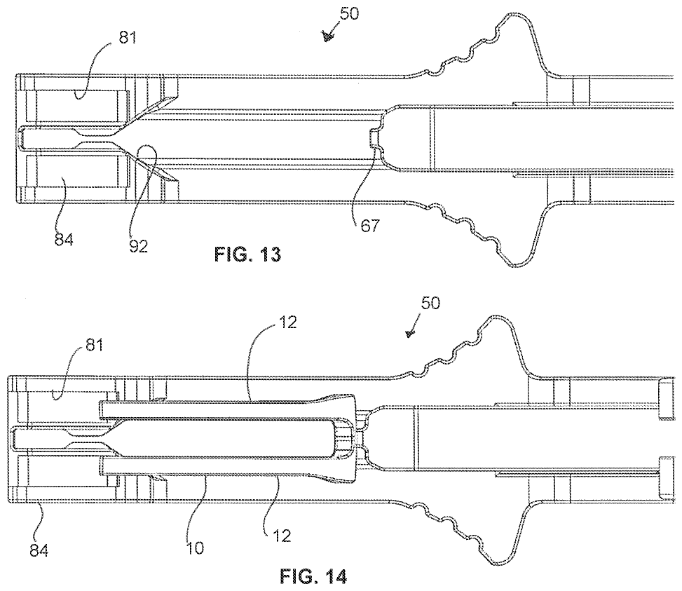

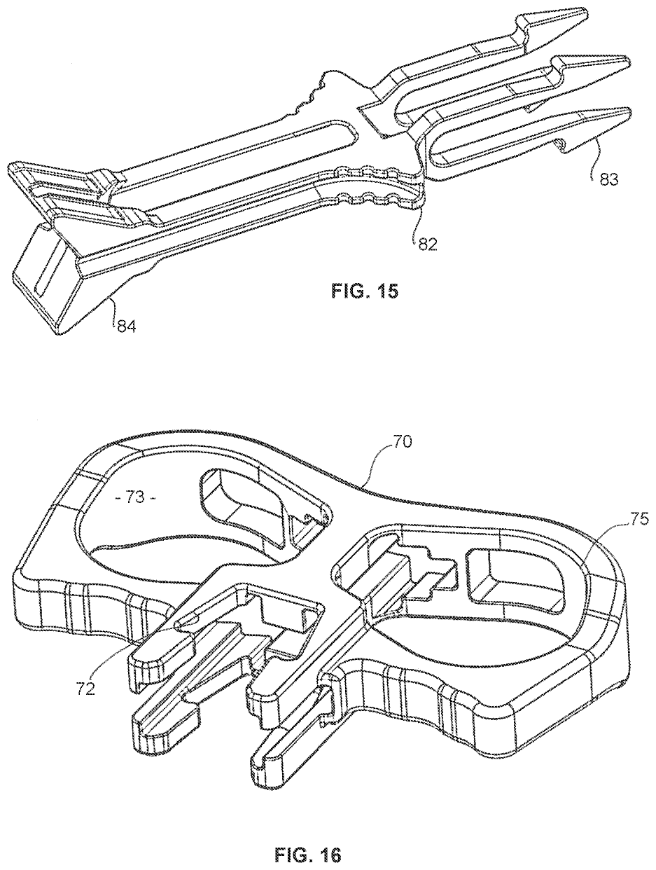

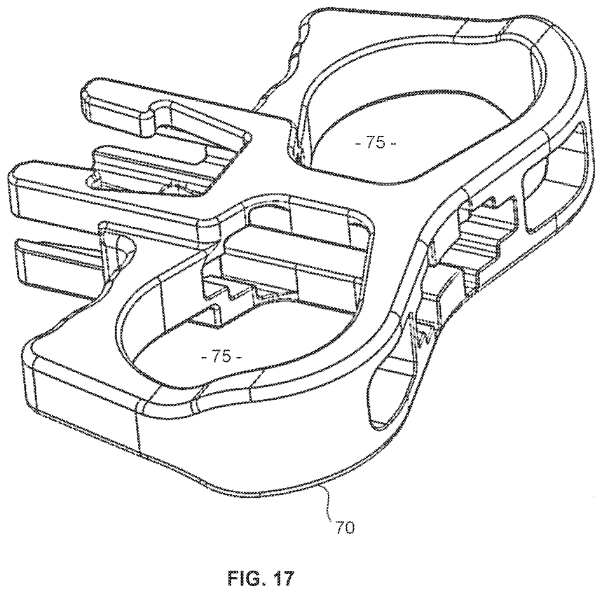

FIG. 11 shows that the selected staple cartridge 80 is loaded into the ram handle assembly. FIG. 12 shows the inserter ready to bring the staple legs into a parallel position. FIG. 13 shows the inserter in an activated position as described. After the bone segments to be fused are prepared for receiving the compression staple 10, the user presses the ram handle 90 using the palm of the hand while engaging the finger holes 72 of the ram housing 70 like a syringe to open the staple legs 12 to the parallel position ready for implantation. The staple is fabricated in the closed (converging legs) shape and is mechanically deformed by the inserter 50 during use to induce the superelastic shape memory properties to compress bone segments and facilitate osteosynthesis. The ram is moved relative to the ram housing by first pressing inward on the flanges of the ram rails to push the rails inward and disengage the ram housing relative to the ram rail stop. Then the user presses against the ram tamp area and draws the housing upward using the inner upper surface 73 of the finger holes 75 and drawing the ram housing 70 upward using the inner upper surface of the finger holes to the point that the fingers meet the bottom surface of the curved stop 63. FIGS. 14 and 15 show the staple inserter aligned with the drill guide handle from differing views and in FIGS. 16 and 17, the system is shown in the positions in which the staple is pushed or tamped into pre-drilled holes.

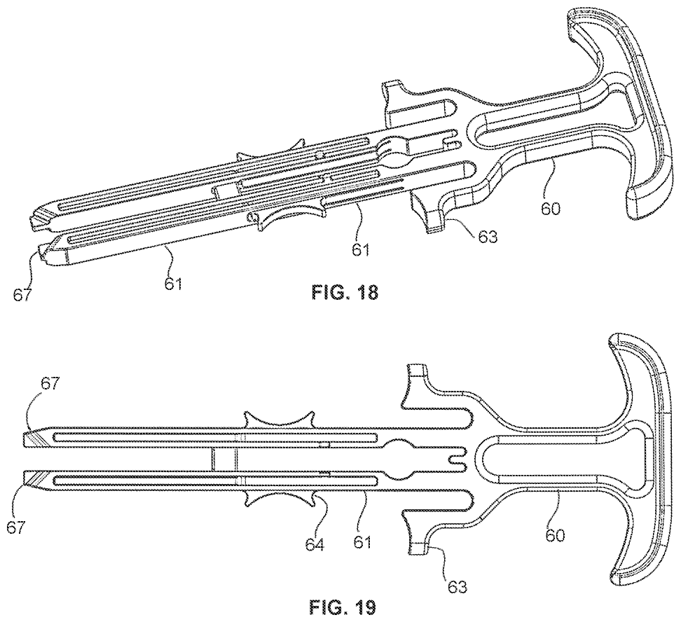

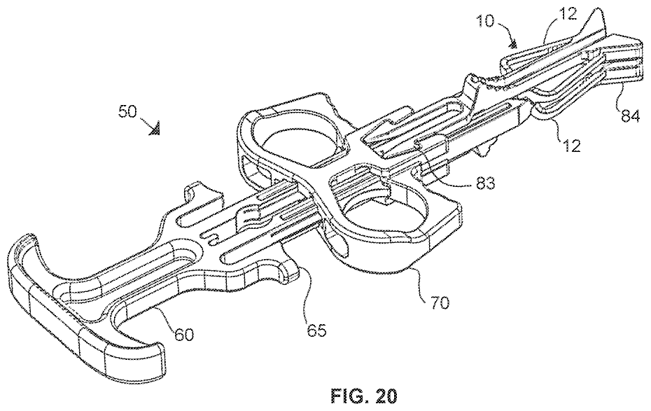

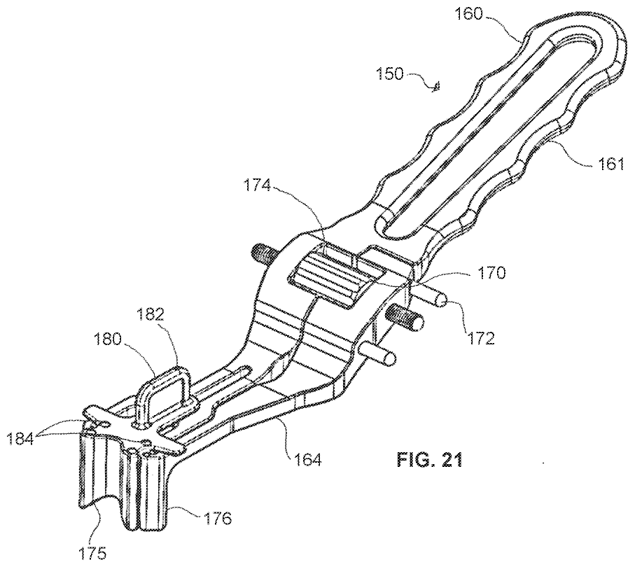

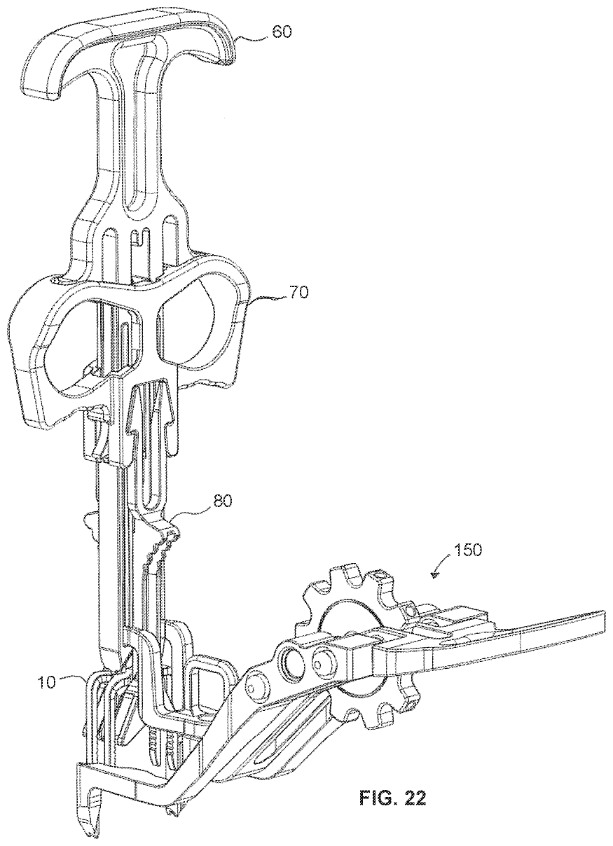

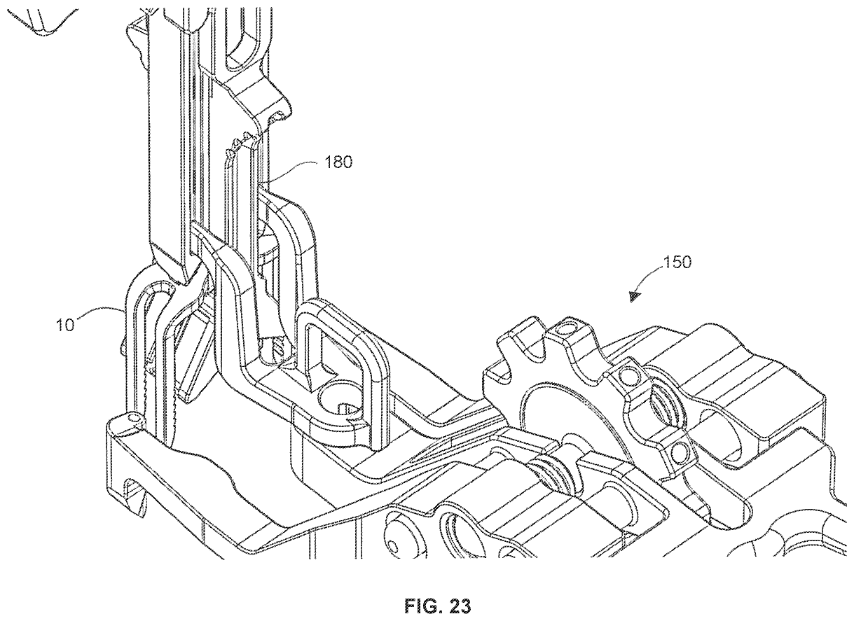

FIGS. 18 and 19 show differing views with the staple inserter assembly having the ram in a final position in the ram handle housing as is described as follows. The ram/ram housing can be further compressed vertically by placing the fingers on the bottom exterior surfaces of the finger holes 74 and compressing the device further in a vertical direction. The curved stops pass through slots in the finger holes allowing this motion to take place, and the staple bridge engages the transverse medial shoulders 92 on the staple clip legs which spreads these legs apart to fully disengage the staple from the inserter. FIG. 20 shows the staple inserter assembly and system when the staple is impacted into the bone, and FIG. 21 shows the system as the inserter and drill guide handle are removed, leaving the staple in place.

Due to the inertial forces as the user squeezes the syringe mechanism, a mechanical lock assembly may be used to ensure that the staple 10 is not inadvertently deployed before being inserted into the bones.

At this point, the user inserts the staple 10 into the bone segments to be fused, the mechanical lock is then released to permit staple release by squeezing the ramp component 107 with the index finger. (The lock section of the ramp component 108 deflects inward to deflect the staple clip legs 86 outwardly and to dis-engage the staple through the opening between the legs of the staple dip.)

The staple inserter of the present invention is suitable for manufacture via injection molding but could also be fabricated from other manufacturing techniques such as, but not limited to, machined, 3-d printed or stamped components. The inserter can be fabricated from plastic or metal materials, or a combination of both.

The staple and inserter are configured to accommodate different fixation procedures in the forefoot, midfoot, rearfoot and hand, and the inserter allows implantation of the staple in bone in a surgical procedure so as to apply a compressive force across a division of bone segments for fracture and osteotomy fixation of the hand and foot, including joint arthrodesis and to stabilize and dynamically compress bone fragments to facilitate osteosynthesis.

What is claimed is:

1. A staple inserter and staple, comprising:
a ram having a handle and which is captured in a sliding engagement in a ram housing mechanism being capable of holding a staple having a pair of legs spaced apart a distance a and connected by a bridge member, the ram housing mechanism having a vertical longitudinal axis and the sliding engagement is in the direction of the vertical longitudinal axis and an expander mechanism which is activated by sliding the ram in the ram housing, the ram housing mechanism including a camming surface that is capable of activating the staple by spreading the legs a distance greater than a; and
the ram housing mechanism has a finger hole portion with a first laterally extending finger grip on a first lateral side of the vertical longitudinal axis and a second laterally extending finger grip on a second lateral side of the vertical longitudinal axis whereby the ram housing can be slide upward on the ram using the first and second laterally extending finger grips.

2. A staple inserter as set forth in claim 1, wherein the ram also has a vertical longitudinal axis which corresponds to the vertical longitudinal axis of the ram housing when the ram is engaged in the ram housing and the ram has a with a first laterally extending ram finger grip on a first lateral side of the vertical ram longitudinal axis and a second laterally extending ram finger grip on a second lateral side of the vertical ram longitudinal axis and the first laterally extending ram finger grip provides a first stop to the sliding of the ram in the ram housing when the first laterally extending ram housing finger grip cooperates with the first laterally extending ram finger grip and wherein the first stop corresponds to an activated position of the staple legs.

3. A staple inserter as set forth in claim 2, wherein one or more of the first and second ram finger grips and the ram housing finger grips are curved and the ram housing has a second set of a first laterally extending finger grip and a second laterally extending finger grip which are spaced apart along the longitudinal axis of the ram housing.

4. A staple inserter as set forth in claim 3, wherein the staple inserter further includes a disengagement mechanism to disengage the staple and the second set of a first laterally extending finger grip and a second laterally extending finger grip corresponds to a position in which the disengagement mechanism has been activated.

5. A staple inserter as set forth in claim 4, wherein the disengagement mechanism comprises a set of medial shoulders which is engaged by the bridge member of the staple when the ram is slide in the ram housing so as to disengage the staple from the staple inserter.

6. A staple inserter as set forth in claim 5, wherein the ram housing mechanism further includes a staple clip and the staple is constrained on the staple clip.

7. A staple inserter, comprising:
a staple mount having a set of openable distal legs having laterally opposed activation ramps, a carriage detachably joined to the staple mount and the carriage having a rail with a long axis and a handle mounted in a sliding engagement along the long axis of the rail and the activation ramps being located on either side of the long axis, wherein the handle can be manipulated using two fingers of a user to bias the open ends of a bone staple against the activation ramps so as to the open the bone staple legs to an implantable position.

8. A staple inserter as set forth in claim 7, wherein the carriage is joined to the staple mount by means of a rotatable gripper.

9. A staple inserter as set forth in claim 8, wherein the rail comprises a pair of rails and further includes a detent for the carriage.

10. A staple inserter as set forth in claim 7, wherein the handle includes two finger hole portions which are on lateral sides of the rail and the body further comprises a proximal palm handle.

11. A staple inserter as set forth in claim 7, wherein the inserter includes a first stop corresponding to an activated position of the staple legs and wherein the two finger hole portions each comprise a curved proximal surface having an opening and the first stop comprises a set of laterally extending arms which encounter the fingers of the user through the openings to cause the user to stop drawing the carriage on the rail.

12. A staple inserter as set forth in claim 8, wherein the rotatable gripper is mounted on the handle of the inserter and biased toward the distal end of the staple mount and is constrained to spiral into a tighter contact with the staple mount as the handle is drawn proximally.

13. A staple inserter as set forth in claim 7, wherein the staple mount has a pair of legs which engage a staple during use and the legs can be opened to insert a staple during use.

14. A bone staple system, comprising:
a sterile pack staple cartridge, comprising:
a staple mount having a set of openable distal legs having laterally opposed activation ramps, the staple mount being loaded with a superelastic staple having at least one pair of legs which have a first position which is relaxed and a second position which is activated whereby the staple can be inserted into bone,
a staple mount carriage detachably joined by a rotatable gripper to the staple mount and the carriage having a body with a rail with a long axis and a handle mounted in a sliding engagement along the long axis of the rail and the activation ramps being located on either side of the long axis, wherein the handle can be manipulated by a user to bias the open ends of a bone staple against the activation ramps so as to the open the bone staple legs to an implantable position.

15. A bone staple system as set forth in claim 15, wherein the legs further includes a stop lock for the carriage which comprises a leaf spring having a J-lock that engages the handle and which corresponding to an activated position of the staple legs.

15. A bone staple system as set forth in claim 14, wherein the legs comprises a pair of rails.

16. A bone staple system as set forth in claim 15, wherein the handle includes two finger hole portions which are on lateral sides of the rails and the body further comprises a proximal palm handle.

17. A bone staple system as set forth in claim 16, wherein the two finger hole portions each comprise a curved proximal surface having an opening and the stop lock comprises a set of laterally extending arms which encounter the fingers of the user through the openings to cause the user to stop drawing the carriage on the rail.

19. A bone staple system as set forth in claim 14, wherein the rotatable gripper is mounted on the handle and biased toward the distal end of the staple mount and is constrained to spiral into a tighter contact with the staple mount as the handle is drawn proximally.

20. A bone staple system as set forth in claim 14, wherein the staple mount has a pair of legs which engage a staple during use and the legs can be opened to insert a staple during use.
